(19) United States
(12) Patent Application Publication
(10) Pub. No.: US 2002/0183649 A1
(43) Pub. Date: Dec. 5, 2002

(54) Distributed port pressure monitor

(76) Inventors: Sanford Reich, Providence, RI (US); James Edward Sluetz, North Attleboro, MA (US)

Correspondence Address: FRANCIS L CONTE, 6 PURITAN AVENUE, SWAMPSCOTT, MA, 01907

(21) Appl. No.: 10/160,837

(22) Filed: May 31, 2002

Related U.S. Application Data
(60) Provisional application No. 60/295,748, filed on Jun. 5, 2001.

(51) Int. Cl. A61B 5/00; G01L 15/00

(52) U.S. Cl. 600/561; 600/16; 73/716

(57) ABSTRACT

A pressure monitor includes a housing containing a reservoir and distributed ports. A pressure sensor is mounted inside the housing at the reservoir for measuring pressure in a liquid contained therein. A flexible membrane sealingly closes the ports and transmits external pressure to the internal liquid for measurement by the pressure sensor.

This application claims the benefit of U.S. Provisional Application No. 60/295,748; filed Jun. 5, 2001.

### CROSS REFERENCE TO RELATED APPLICATION

U.S. patent application No. 09/472,708 entitled Dual Pressure Monitor by S. Reich, discloses an implantable primary pressure sensor which measures absolute pressure in a patient, and an implantable remote pressure sensor cooperating therewith for measuring barometric pressure external of the patient. The two pressure sensors are interconnected by a conduit containing a pressure transmitting liquid.

This invention was made with United Stated Government support under Cooperative Agreement No. 70NANB7H3059 awarded by NIST. The United States Government has certain rights in the invention.

### BACKGROUND OF THE INVENTION

The present invention relates generally to pressure sensors, and, more specifically, to implantable pressure sensors.

In the medical field pertaining to living patients, pressure sensing of bodily fluids introduces the additional requirement of patient safety. For example, the measurement of blood pressure must not damage the blood itself or form clots therein which are detrimental to patient health.

Artificial heart pumps are being developed in the exemplary form of a Left Ventricular Assist Device (LVAD) which assists damaged hearts. Typical artificial heart pumps are configured for varying blood flowrate, frequency, and pressure as required to meet the typical demands placed on the heart which change in response to work effort. It is therefore desirable to control the heart pump by sensing blood pressure in the body.

In clinical practice, the tricuspid valve between the right atrium and right ventricle is chosen as the reference level for pressure measurement because this is one point in the circulatory system at which hydrostatic pressure factors caused by body position of a normal person usually do not affect the pressure measurement by more than 1 or 2 mm Hg. The reason for the lack of hydrostatic effects at the tricuspid valve is that the heart automatically prevents significant changes at this point by acting as a feedback regulator of pressure at this point.

For example, if the pressure at the tricuspid valve rises sightly above normal, the right ventricle fills to a greater extent than usual, causing the ventricle to pump more blood more rapidly and therefore to decrease the pressure at the tricuspid valve toward zero mm Hg. Thus all clinical blood pressure measurements are gauge pressure measurements referenced to barometric pressure and independent of barometric pressure, and referenced to the tricuspid valve level.

Since the heart pump is preferably fully implanted inside a patient, blood pressure must be also measured inside the body for controlling the pump. However, since it is not practical to directly measure blood pressure at the tricuspid valve, a suitable alternate pressure source must be provided.

In the previous development disclosed in the above-identified patent application the primary and remote pressure sensors are interconnected by a liquid carrying conduit. Accordingly, a pressure being detected by the remote pressure sensor is affected by the hydrostatic pressure in the interconnecting conduit which can substantially affect the reference pressure, and therefore requires suitable correction during operation.

Accordingly, it is desired to provide an implantable pressure monitor for referencing outside barometric pressure for controlling an implanted heart pump.

### BRIEF SUMMARY OF THE INVENTION

A pressure monitor includes a housing containing a reservoir and distributed ports. A pressure sensor is mounted inside the housing at the reservoir for measuring pressure in a liquid contained therein. A flexible membrane sealingly closes the ports and transmits external pressure to the internal liquid for measurement by the pressure sensor.

### BRIEF DESCRIPTION OF THE DRAWINGS

The invention, in accordance with preferred and exemplary embodiments, together with further objects and advantages thereof, is more particularly described in the following detailed description taken in conjunction with the accompanying drawings in which:

FIG. 1 is an schematic representation of a human heart inside the relevant portion of a human body, including a heart assist pump joined to the heart by a primary pressure monitor cooperating with a reference pressure monitor implanted subcutaneously in accordance with an exemplary embodiment of the present invention.

FIG. 2 is a sectional view of the primary pressure monitor illustrated in FIG. 1 and taken along line 2-2.

FIG. 3 is an elevational sectional view of the reference pressure monitor illustrated in FIG. 1 in accordance with an exemplary embodiment.

FIG. 4 is a partly sectional isometric view of the reference pressure monitor illustrated in FIGS. 1 and 3 in conjunction with a flowchart representation of its method of assembly and use in accordance with preferred embodiments.

### DETAILED DESCRIPTION OF THE INVENTION

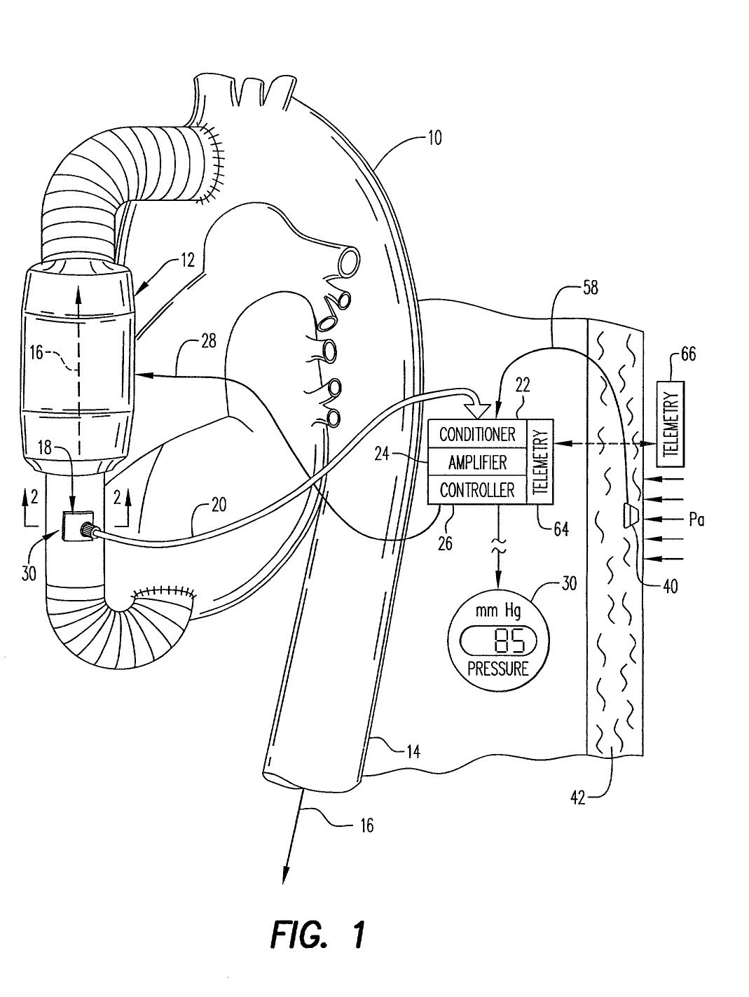

Illustrated schematically in FIG. 1 is a human heart 10 inside the relevant portion of a living patient or body to which a Left Ventricular Assist Device (LVAD) or heart pump 12 is joined. The heart pump may take any conventional form and is sutured in the patient, in this case between the left ventricle of the heart and the main artery or aorta 14 for assisting in pumping fluid or blood 16.

A primary pressure monitor 18 joins the heart pump in flow communication with the left ventricle for carrying blood through the pump while simultaneously measuring pressure thereof. The pressure monitor is operatively joined by an electrical cable 20 to a conventional signal conditioner or processor 22 which in turn is operatively joined to an amplifier 24 and electrical controller 26 which controls operation of the heart pump including its flowrate, frequency, and pumping pressure.

The controller 26 may take any conventional form, and is operatively joined also to the heart pump by another electrical cable 28 for controlling pumping of the blood though the pump in response to measured pressure from the pressure monitor. The controller is suitably configured for controlling blood flow though the pump into the aorta, and may optionally be joined to a suitable remote indicator 30 for permitting external visual observation of the measured blood pressure which may be expressed in any suitable unit, such as millimeters of mercury (mm Hg).

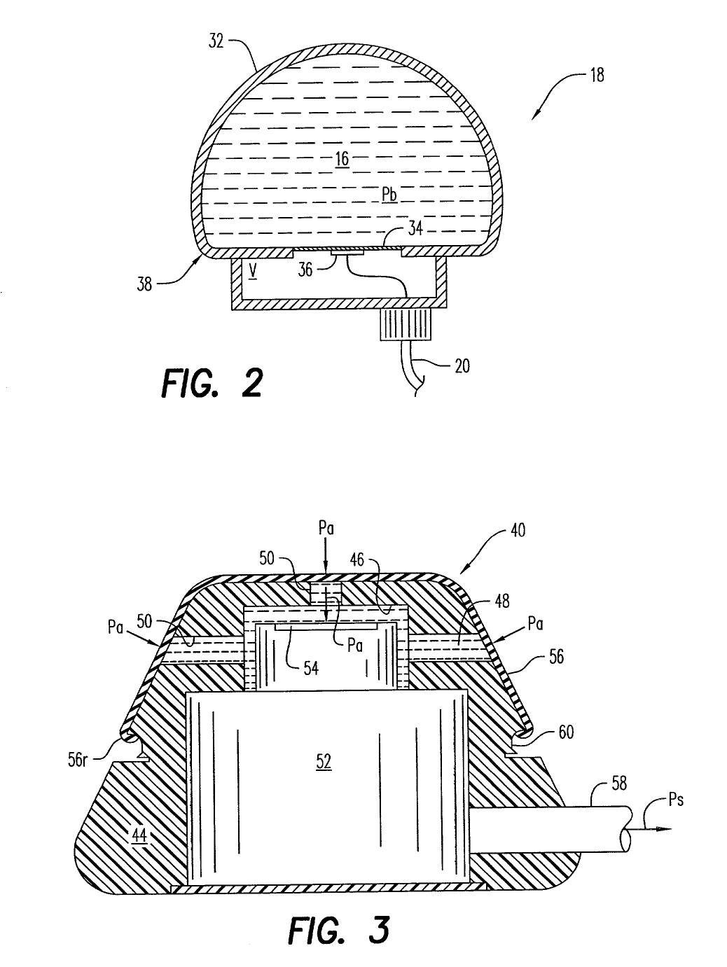

The primary monitor 18 is illustrated in more particularity in FIG. 2 and includes a cannula tube 32 through which the blood fluid 16 is channeled during operation of the pump. Since the fluid in this exemplary embodiment is blood, the tube is preferably formed of a hemo-compatible material, such as titanium, having proven benefits for carrying blood flow without incompatibility therewith. The tube is preferably smooth and seamless with a relatively thin wall.

The tube is primarily annular or cylindrical and includes a flat wall section having an opening in which is mounted a flexible primary diaphragm 34 which adjoins or bounds in part the fluid carried through the tube. The diaphragm is preferably planar and flat and may be made of thin titanium of about five mils (0.1 3 mm) thickness for being flexible under blood pressure.

Means in the exemplary form of a primary gauge 36 adjoin the diaphragm 34 on the outer surface thereof for measuring flexure of the diaphragm under pressure from the blood inside the tube. The signal processor 22 is operatively joined to the primary gauge to determine the fluid pressure of the blood inside the tube as measured from flexure of the diaphragm caused by the fluid pressure.

In a preferred embodiment, the primary gauge 36 includes a plurality of conventional strain gauges mounted to the diaphragm for measuring strain therein due to flexure of the diaphragm under pressure. The strain gauges may take any conventional form and are typically adhesively bonded or joined by sputtering to the outer surface of the diaphragm in any suitable configuration, such as four in-line strain gauges.

The strain gauges are suitably electrically joined to the processor 22 for producing an electrical voltage signal as the diaphragm is elastically deformed under pressure. The pressure of the fluid inside the tube creates longitudinal and circumferential strain in the thin diaphragm as it flexes which is indicative of the pressure of the blood inside the tube 32.

Since blood pressure is being measured by induced strain in the diaphragm 34, that strain is based on the differential pressure acting across the diaphragm. Since the diaphragm is implanted in a living body, the nominal pressure therein is variable and unknown.

Accordingly, it is desired to provide a stable reference pressure inside the body for use in more accurately determining blood pressure. For in vivo conditions, a vacuum is considered to be a stable and practical reference since a vacuum may be maintained at a constant value, or vacuum pressure, and will not vary as temperature changes inside the body.

By providing a vacuum outside the diaphragm 34, the blood pressure measured by the primary gauge 36 is substantially an absolute pressure measurement which does not change as barometric pressure outside the body changes.

Accordingly, a primary cell 38 is fixedly joined to the tube 32 outside the primary diaphragm 34 for providing an enclosed chamber therearound which may be suitably evacuated to a suitably low vacuum pressure V.

However, as indicated above, clinical blood pressure measurements are preferably gauge pressure which are referenced to barometric pressure and are independent therefrom. Since barometric pressure changes due to weather high and low pressures and due to altitude above sea level, such changes are not reflected in the absolute pressure measured by the primary gauge 36.

By introducing a vacuum in the primary cell 38, the pressure difference across the diaphragm 34 is increased and the measured pressure of the blood 16 is an absolute pressure relative to the degree of vacuum provided in the cell. Since the primary cell is under vacuum, there is no opposing pressure on the diaphragm 34 which affects flexure of the diaphragm for more accurately determining the blood pressure inside the tube.

Furthermore, the vacuum inside the cell 50 does not change pressure therein due to changes in temperature at the primary cell as body temperature changes. Accordingly, the vacuum provides a stable reference pressure from which an accurate measurement of the blood pressure may be obtained by diaphragm flexure.

In accordance with another feature of the present invention, a non-blood pressure inside the body must be discovered which is closely related to barometric pressure and independent of hydrostatic or other pressures in the body. Such a non-blood pressure must also be capable of measurement in vivo inside the body, yet must also be subject to calibration based on barometric pressure outside the body.

These objectives may be met by using a remote pressure monitor 40 illustrated in FIGS. 1 and 3. As shown in FIG. 1, the remote monitor 40 is preferably implanted subcutaneously below the skin 42 of the patient for being responsive to barometric or atmospheric pressure Pa exerted on the skin. The remote pressure monitor 40 may therefore be used to provide a reliable reference pressure for the absolute pressure measured by the primary pressure monitor 18 for obtaining gauge pressure in conjunction therewith which may be used for controlling operation of the pump 12.

More specifically, the remote monitor 40 illustrated in FIG. 3 is sized and configured to be as small as possible for implantation below the skin for providing an accurate indication of external barometric pressure while minimizing damage to living tissue. The remote monitor includes a small rigid housing 44 which may be made of any suitable material, such as plastic in the form of polysulfone or Delrin for example. The housing includes a central chamber or reservoir 46 for holding an incompressible liquid 48, such as saline water which is biocompatible with living bodies.

The housing includes a plurality of distributed inlet ports 50 therethrough which are each disposed at one end in flow communication with the common reservoir and at opposite ends are exposed at the outer surface of the housing.

A remote pressure sensor 52 is suitably mounted inside the housing 44 at the reservoir 46 for measuring pressure in the liquid contained therein. The pressure sensor 52 may have any conventional form and is preferably as small as possible for correspondingly permitting the housing 44 to be as small as possible.

In a preferred embodiment, the pressure sensor 52 includes a flat pressure sensing diaphragm 54 disposed inside the reservoir for measuring pressure of the liquid contained therein exerted upon the diaphragm. The entire remote pressure sensor 52 is preferably hermetically sealed for protecting all of its working electrical components, with the diaphragm 54 being exposed to the liquid. Pressure exerted on the diaphragm effects strain therein which is suitably measured for indicating the corresponding pressure exerted on the diaphragm.

A suitable remote pressure sensor is of the type identified as HKM-191-13375T as manufactured by Kulite Semiconductor Products, In.c, Leonia, N.J. However, any type of small pressure sensor may be used for detecting pressure of the liquid contained in the reservoir.

In order to protect the fragile diaphragm 54 of the remote sensor 52, the surrounding housing 44 is suitably rigid, and the ports 50 are distributed around the housing for providing redundant channels for communicating external pressure through the liquid and to the diaphragm. Accordingly, a flexible membrane 56 is suitably joined to the housing to sealingly close the several ports 50 and trap and retain the liquid inside the reservoir and ports.

The flexible membrane may be formed of silicone having a suitably low durometer of about 20A to about 40A for being supple and flexible to permit unobstructed transfer of the external pressure around the membrane into the liquid contained inside the housing which transfers the pressure to the diaphragm 54. The membrane is also substantially water impermeable and provides an effective seal for retaining the reservoir and ports completely filled with the liquid.

Since the remote housing 44 is small for implantation, the ports 50 therein are correspondingly smaller and thus subject to external contact forces on the skin which might close any one or more of the ports and prevent pressure transfer therethrough. Accordingly, the several ports 50 are preferably distributed in different directions in the housing to reduce the likelihood that all of the ports might temporarily be closed during use.

For example, the skin in the region of the implanted remote pressure monitor 40 may be subject to tight clothing or weight pressure due to the patient sleeping and rolling in bed which might block any one of the ports. Distributing the ports increases the likelihood that at least one of the ports remains unblocked for detecting barometric pressure at the patient's skin, with any blocked ports not adversely affecting the barometric pressure measurement.

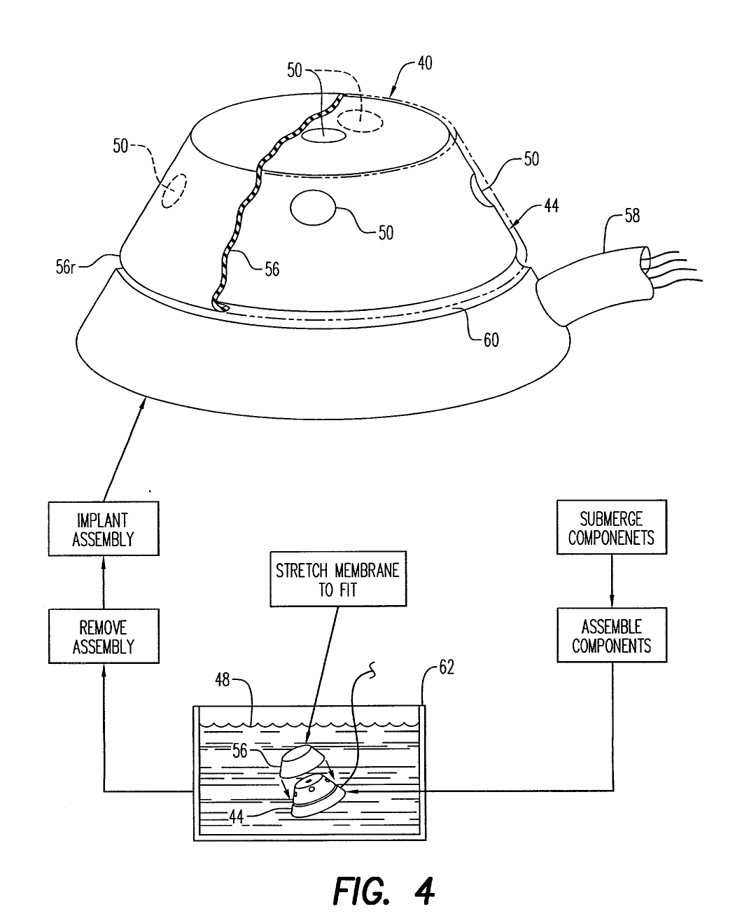

As illustrated in FIGS. 3 and 4, the several ports 50 are distributed at least in part laterally around the sides of the housing 44, with preferably some of the ports being distributed laterally in a common horizontal plane, and another one of the ports being disposed perpendicularly to that plane for maximizing the difference in orientation or direction of the several ports.

In a preferred embodiment, four of the ports 50 are circumferentially spaced apart substantially equiangularly around the housing 44 at about 90° apart and therefore face laterally outwardly. A fifth one of the ports 50 is preferably centered in the top of the housing between the four lateral ports in the common plane and faces perpendicularly outwardly or upwardly therefrom. In this way, five ports are provided which face in five different directions.

The top port would typically be implanted directly under the patient's skin to more directly measure barometric pressure thereat, and would therefore be most likely subject to being blocked by contact pressure against the skin. The four lateral ports face generally parallel to the surface of the skin and are less likely to be blocked by surface contact force, especially in view of their four different orientations.

As shown in FIG. 3, the exemplary embodiment of the remote pressure sensor 52 includes a narrow cylindrical tip which is sized at the diaphragm 54 contained therein to provide a lateral gap or annulus in the reservoir 46 circumferentially surrounding the pressure sensor tip, and a top gap atop the diaphragm and below the top port 50.

The four lateral ports 50 are preferably horizontally aligned or disposed in direct flow communication with the lateral annulus of the reservoir, and the top port is preferably vertically aligned or disposed in direct flow communication with the top gap of the reservoir. In this way, the reservoir 46 surrounds the tip of the pressure sensor and all five ports 50 have unobstructed flowpaths to the diaphragm 54 for transmitting the external pressure forces on the membrane to the diaphragm 54.

In the exemplary embodiment illustrated in FIG. 3, the pressure sensor 52 includes a narrow cylindrical tip containing the diaphragm 54, and a larger or broader cylindrical base mounted inside a bottom end of the housing 44. The base includes an electrical cable 58 operatively joined to the diaphragm 54, and extends laterally through the housing 44. The cable 58 is joined to the signal processor 22 as illustrated in FIG. 1 for providing an electrical pressure signal Ps indicative of the external atmospheric or barometric pressure Pa. The barometric pressure is transferred through the patient's skin to the flexible membrane 56, and in turn to the liquid contained in the reservoir 46 and is then exerted on the pressure sensing diaphragm 54 which in turn effects the electrical pressure signal Ps indicative of the measured pressure.

As shown in FIGS. 3 and 4, the membrane 56 is preferably a unitary element which surrounds the housing 44 at least in part to sealingly close all of the several ports 50. The membrane 56 preferably has an inverted generally cup shape, open at the bottom end, closed at the opposite top end, and additionally closed therearound for effecting a common annular sidewall.

In the preferred embodiment illustrated in FIGS. 3 and 4, the housing 44 is frustoconical with a narrower diameter top and larger diameter base, with a conically tapered sidewall therebetween. The several ports 50 are distributed in both the housing top and sidewall, and the membrane 56 covers the top and sidewall over the ports.

Surrounding the conical sidewall of the housing 44 is an annular perimeter notch 60 provided for engaging a perimeter rim 56r of the membrane 56 for retaining the membrane on the housing to cover the several ports. The membrane 56 may be slightly undersized compared with the frustoconical top end of the housing above the notch 60 so that the membrane may be slightly stretched during assembly over the housing, with the rim 56r being slightly stretched to engage the notch 60 for retention therein. Residual hoop tension force in the membrane rim 56r effects a suitable seal with the notch to prevent escape of the liquid from the ports and reservoir during operation.

As shown in FIG. 3, the frustoconical housing 44 preferably includes smooth and rounded corners joining its annular sidewall with both the top and base of the housing for eliminating sharp corners in the housing for reducing the likelihood of any damage to the skin tissues due to the presence of the implanted pressure monitor.

Also shown in FIG. 3 is a preferential location of the several ports 50 at the top of the housing. The notch 60 is disposed centrally between the housing top and base, and the ports 50 are distributed in the sidewall between the notch and housing top. In this way, the side ports 50 are disposed in close proximity and cooperation with the top port to locally provide differently facing ports and common flow communication with the central reservoir for independently transmitting external pressure through the membrane into the liquid in the reservoir for measurement by the internal pressure sensor 52.

The resulting remote pressure monitor 40 is relatively small with rounded contours for subcutaneous implantation. The distributed ports are disposed closely adjacent to the skin surface for independently experiencing barometric pressure. The detected barometric pressure is then transmitted electrically to the common signal processor 22 to provide a reference pressure for the absolute pressure measured by the primary pressure monitor 18.

FIG. 4 illustrates schematically a preferred method of assembling the remote pressure monitor 40. The housing 44 and membrane 56 are separately submerged in a container or pool 62 of the liquid 48. This is preferably done at the time of implantation in the patient.

The liquid in the pool is then permitted to fill completely the internal reservoir and ports of the housing submerged in the pool. The membrane is then assembled over the housing while both are submerged in the pool to trap the liquid inside the reservoir and ports. And, the so assembled pressure monitor is then removed from the pool and then implanted in the patient.

This assembly procedure maintains sterility of the pressure monitor components, including saline water captured therein. And, no water is lost from the assembled monitor in the short interval in which it is implanted into the patient.

Assembly of the monitor components is quite simple inside the pool 62 in view of the simple frustoconical configuration of the housing 42 and corresponding cup-like shape of the unitary membrane 56. The membrane may be simply stretched when submerged in the pool to enclose the top of the housing while engaging the membrane rim 56r with the perimeter notch 60 for effecting sealed retention thereto.

The simplified configuration of the remote pressure monitor permits not only the simple assembly thereof but the correspondingly simple implantation into the patient. The housing reservoir 46 and ports 50 are simply filled with the water 48 in the pool 62, with the membrane 56 then being used to sealingly contain the water therein. The pressure monitor 40 is then implanted subcutaneously in the patient for use as a reference pressure monitor for measuring external barometric pressure exerted on the patient's skin.

The primary pressure monitor 18 is also implanted in the patient for measuring absolute pressure at a suitable location. And, both the primary and reference pressure monitors are then used to collectively obtain a gauge pressure value in which the absolute pressure is referenced by the measured barometric pressure, with the gauge pressure being the difference therebetween.

In the preferred embodiment illustrated in FIG. 1, the LVAD 12 is also implanted in the heart of the patient. The primary pressure monitor 18 is disposed at the inlet of the pump 12 to measure absolute pressure of the blood thereat. The reference pressure monitor 40 is then used to measure barometric pressure at the skin of the patient. And, the gauge pressure value obtained by the difference between the primary and reference pressure monitor signals is used to control operation of the pump 12.

Electrical signals from both the primary pressure monitor 18 and reference pressure monitor 40 illustrated in FIG. 1 are provided to the signal processor or conditioner 22. These signals are suitably calibrated with offset and gain adjustments as required for providing accurate pressure measurements from the two monitors. The two pressure monitors may be calibrated at the time of the manufacture or initial implantation; and may be recalibrated as desired after in vivo implantation for each patient.

In vivo recalibration may be simply effected without additional surgery by introducing an internal telemetry circuit 64 in conjunction with the signal processor. Telemetry circuits are conventionally known and permit airborne communication of calibration information with an external telemetry circuit 66 configured for communication with the internal telemetry circuit 64.

The raw electrical signals from the primary and remote pressure monitors 18, 40 may then be calibrated in the signal processor for improving accuracy thereof. The calibrated barometric pressure signal from the remote monitor 40 is then subtracted in the signal conditioner from the calibrated absolute pressure signal from the primary monitor 18 to obtain the desired gauge pressure signal. The gauge signal is then suitably used in the pump controller 26 for controlling operation of the pump 12 in response to the measured gauge pressure.

The pressure monitor 18 may be used to advantage in controlling the heart pump 12 by implanting the heart pump 12 and tube 32 in series in the heart fully inside the patient. The remote monitor 40 is preferably implanted subcutaneously below the skin of the patient for being responsive to the barometric pressure exerted on the skin.

The pressure exerted on the skin is atmospheric pressure Pa, which is zero gauge pressure assuming that there is no tight clothing confining that particular skin location, or no object of significant weight exerting a force on that area of skin. The rigid housing of the remote monitor is designed to minimize the effect of such extraneous external forces.

In general, pressure transmitted to subcutaneous tissue from its surroundings is the total tissue pressure (TTP). The TTP is the algebraic sum of the following two pressures:

(1) Interstitial fluid pressure (IFP): This pressure from the free fluid in the surrounding minute tissue spaces, as opposed to the surrounding interstitial fluid gel that normally constitutes 99% of the tissue fluid content. This pressure is independent of hydrostatic pressure because of the protein structure that creates the interstitial gel fluid structure. The IFP is normally negative (−) 2 mm Hg and typically ranges from −3 to −1 mm Hg when measured using a hypodermic needle inserted subcutaneously; and

(2) Solid tissue pressure (STP): This pressure represents the force exerted by the solid elements of the tissues upon each other. These forces cause the cells and other solid structures to resist compression when negative pressure in the interstitial fluid sucks the solid structure against each other. It also causes much of the transmission of atmospheric pressure from the skin into the subcutaneous tissue.

When the remote monitor 40 is implanted subcutaneously, an encased pocket of dense connective tissue will form therearound in approximately one month. The TTP may be slightly positive, but should be a relatively small and constant offset pressure relative to atmospheric pressure. The TTP value may go through some transition during the first month following implantation.

The anticipated constant offset pressure from subcutaneous implantation of the probe may increase by several mm Hg if a significant edema develops. When a significant edema occurs, the interstitial pressure may be as high as +6 mm Hg versus −2 mm Hg in the normal state.

A significant edema is said to be a pitting edema because one can press the thumb against the tissue area and push the fluid out of the area. When the thumb is removed, a pit left in the skin for a few seconds until the free fluid flow back from the surrounding tissues. A significant edema may result from many serious conditions including heart failure, kidney failure, bacterial infections, cancer, liver disease, and loss of plasma proteins from significant skin burns and wounds.

However, the TTP closely tracks barometric pressure in the normal physiological state and increases in value in disease or injury states that are easily detected by the presence of an edema. Under normal physiological states, the TTP pressure variations are expected to be within required accuracy of the primary pressure sensor 30, e.g., +/−1.5 mm Hg.

Under abnormal physiological states, the TTP pressure variations are expected to shift to about three times the minimum expected primary sensor accuracy in the positive pressure side. This abnormal positive shift in barometric reference pressure will cause the gauge pressure to decrease by the same amount and be perceived as a decrease in primary pressure.

Since the remote monitor 40 illustrated in FIG. 3 is preferably implanted subcutaneously, it is not directly exposed to the ambient pressure Pa, and the reference pressure Pr exerted inside the monitor 40 may not be exactly equal to the barometric pressure. The reference pressure Pr inside the probe is thusly a combination of the external barometric pressure Pa and local internal pressures within the skin. The actual difference in the barometric pressure and the reference pressure may be determined during calibration, with a suitable offset factor being determined therefor.

Accordingly, the remote monitor is preferably calibrated by comparing separately measured barometric pressure with the pressure measured by the remote monitor, and determining any correction or offset factor which may be introduced into the signal conditioner for improving accuracy.

The reference membrane 56 illustrated in FIG. 3 is preferably slightly water permeable for automatically relaxing following transient changes in barometric pressure. Silicone rubber is a preferred choice for the reference membrane 56 since it permits slow water diffusion between the skin tissue and saline liquid 48 when the membrane is placed under external pressure.

This is particularly useful as barometric pressure changes due to weather, or due to elevation changes as a patient travels between sea level and the mountains. As the reference membrane 56 is deflected or stressed under changes in barometric pressure, it will slowly relax to an unstressed state as water diffuses therethrough over one or more days. In this way, the remote monitor is self-nulling to changes in barometric pressure, which correspondingly ensures that the pressure monitor is referenced to the local barometric pressure.

A particular advantage of the distributed port pressure monitor 40 implanted under the skin as illustrated in FIG. 1 is the independent detection of external barometric pressure by the several ports notwithstanding contact force blockage of any one or more, but not all, of the ports. Since the remote monitor 40 is self contained with a small volume of saline water therein, pressure measured thereby is closely related to the barometric pressure on the patient's skin, and is independent of hydrostatic or other pressures in the body. The implanted remote monitor 40 measures pressure inside the body indicative of external barometric pressure, and is readily subject to calibration based on the barometric pressure outside the body.

In this way, the remote monitor 40 increases accuracy of measuring barometric pressure outside the patient notwithstanding its implanted location. The calibrated measurement of barometric pressure is then used in conjunction with the absolute pressure measured by the primary pressure monitor 18 for providing an accurate representation of gauge pressure of the blood entering the heart pump 12, and thusly improved control of the heart pump may be obtained.

While there have been described herein what are considered to be preferred and exemplary embodiments of the present invention, other modifications of the invention shall be apparent to those skilled in the art from the teachings herein, and it is, therefore, desired to be secured in the appended claims all such modifications as fall within the true spirit and scope of the invention.

Accordingly, what is desired to be secured by Letters Patent of the United States is the invention as defined and differentiated in the following claims in which we claim:

1. A pressure monitor comprising:
a housing including a reservoir for holding a liquid, and plurality of distributed ports disposed in flow communication with said reservoir and exposed at an outer surface of said housing;
a pressure sensor mounted inside said housing at said reservoir for measuring pressure in said liquid; and
a flexible membrane joined to said housing to sealingly close said ports.

2. A monitor according to claim 1 wherein said ports are distributed in different directions in said housing.

3. A monitor according to claim 2 wherein said ports are distributed at least in part laterally around said housing.

4. A monitor according to claim 3 wherein some of said ports are distributed laterally in one plane, and another one of said ports is disposed perpendicularly to said plane.

5. A monitor according to claim 3 wherein said laterally distributed ports are substantially equiangularly spaced apart.

6. A monitor according to claim 3 wherein four of said ports are circumferentially spaced apart around said housing at about 90° apart and face laterally outwardly; and a fifth port is centered in said housing between said four ports and faces perpendicularly outwardly therefrom.

7. A monitor according to claim 3 wherein said pressure sensor includes a pressure sensing diaphragm disposed inside said reservoir, and said pressure sensor is sized at said diaphragm to provide a lateral annulus in said reservoir circumferentially surrounding said pressure sensor, and a top gap atop said diaphragm.

8. A monitor according to claim 7 wherein said lateral ports are disposed in flow communication with said lateral annulus; and another one of said ports is disposed in flow communication with said top gap.

9. A monitor according to claim 7 wherein said pressure sensor includes a narrow tip containing said diaphragm, and broader base mounted inside a bottom end of said housing, and said base includes an electrical cable operatively joined to said diaphragm and extending laterally through said housing to provide an electrical pressure signal indicative of pressure measured by said diaphragm through said liquid.

10. A monitor according to claim 3 wherein said membrane is unitary and surrounds said housing to sealingly close all said ports.

11. A monitor according to claim 10 wherein said membrane has a generally cup shape open at one end, closed at an opposite end, and additionally closed therearound.

12. A monitor according to claim 11 wherein:
said housing is frustoconical with a narrow diameter top and a larger diameter base, and a tapered sidewall therebetween;
said ports are distributed in both said housing top and sidewalls; and
said membrane covers said top and sidewall over said ports.

13. A monitor according to claim 12 wherein:
said housing further includes an annular notch surrounding said sidewall below said ports therein; and
said membrane further includes a rim engaging said notch for retaining said membrane on said housing.

14. A monitor according to claim 12 wherein said frustoconical housing includes smooth and rounded corners joining said sidewall with both said top and base.

15. A monitor according to claim 14 wherein said notch is disposed centrally between said housing top and base, and said ports are distributed in said sidewall between said notch and housing top in cooperation with said port in said housing top to locally provide differently facing ports in common flow communication with said reservoir for independently transmitting external pressure through said membrane into said liquid in said reservoir for measurement by said pressure sensor.

16. A monitor according to claim 15 further comprising said liquid disposed in said reservoir.

17. A method of assembling said pressure monitor according to claim 3 comprising:
separately submerging both said housing and membrane in a pool of said liquid;
permitting said liquid to fill said reservoir and ports while submerged in said pool;
assembling said membrane over said housing while submerged in said pool to trap said liquid inside said reservoir and ports; and removing said assembled pressure monitor from said pool.

18. A method according to claim 17 further comprising stretching said membrane in said pool to engage a perimeter notch in said housing for sealed retention thereto.

19. A method of using said pressure monitor according to claim 3 comprising:
filling said reservoir and ports with said liquid, with said membrane sealingly containing said liquid therein;
implanting said pressure monitor as a reference pressure monitor subcutaneously in a living patient for measuring external barometric pressure;
implanting a primary pressure monitor in said patient for measuring absolute pressure therein; and
using said reference and primary pressure monitors to collectively obtain a gauge pressure value.

20. A method according to claim 19 further comprising:
implanting a left ventricular assist device in the heart of said patient;
using said primary pressure monitor to measure absolute pressure at an inlet to said device;
using said reference pressure monitor to measure barometric pressure at the skin of said patient; and
using said gauge pressure value to control operation of said device.
